(19) United States
(12) Patent Application Publication
(10) Pub. No.: US 2022/0409090 A1
(43) Pub. Date: Dec. 29, 2022

(54) OPPORTUNISTIC SONAR MONITORING OF VITAL SIGNS

(71) Applicant: Google LLC, Mountain View, CA (US)

(72) Inventors: Dongeek Shin, Santa Clara, CA (US); Qiuyue Xue, Seattle, WA (US); Shwetak Patel, Seattle, WA (US)

(73) Assignee: Google LLC, Mountain View, CA (US)

(21) Appl. No.: 17/360,999

(22) Filed: Jun. 28, 2021

(51) Int. Cl. A61B 5/097; A61B 5/00; G01S 7/00

(52) U.S. Cl. CPC A61B 5/097; A61B 5/6898; A61B 5/749; A61B 5/742; A61B 5/0015; A61B 5/0004; G01S 7/003; A61B 2562/0219

(57) ABSTRACT

Various arrangements for sonar-based vital sign monitoring are presented herein. A mobile device may be determined to be stationary. In response, sonar-based movement sensing can be activated. Sonar data can then be captured in response to activating the sonar-based movement sensing. A breathing pattern can be detected in the sonar data and used to collect respiration data about a user.

### BACKGROUND

Respiratory Rate (RR) is an important vital sign. An abnormal respiratory rate has been shown to be an important indicator of serious health-related events such as respiratory failure and cardiac arrest. An RR higher than 20 BPM (Breaths Per Minute) may be considered tachypnoea and can be caused by a variety of clinical and non-clinical factors (e.g., stress, infection, cardiopulmonary disorders). An RR lower than 12 BPM may be considered bradypnoea and can be caused by various clinical and non-clinical factors (e.g., volitional respiration reduction through meditation or breath-work, medication side effects, sleep apnea). Therefore, monitoring a person's respiratory rate can offer insights into the person's respiratory health and potentially predict the onset or exacerbation of health problems. However, most persons do not have access to dedicated equipment to monitor their breathing. Therefore, there is a need to monitor a person's breathing without requiring a dedicated device.

There are several possible contactless sensing methods for respiratory monitoring, such as radar-based and camera-based. However, such arrangements may obtain the user's respiratory rate using active sensing arrangements. Such active sensing arrangements can require the user to manually start an application or initiate a system and pay attention during a measuring time period. Such active sensing arrangements require significant user effort and it may be difficult for a user to remember to use it. As a result, an active sensing application or device for daily health monitoring can be expected to have a high drop-out rate. Further, when a user is aware that a device is measuring their respiratory rate, they may consciously or unconsciously adjust their breathing—thereby resulting in an altered measurement.

### SUMMARY

Various embodiments are described related to a method for sonar-based respiration monitoring. In some embodiments, a method for sonar-based respiration monitoring may be described. The method may comprise determining, by a mobile device, that the mobile device may be stationary. The method may comprise, in response to determining that the mobile device may be stationary, activating, by the mobile device, sonar-based movement sensing. The method may comprise capturing, using a sonar sensor of the mobile device, sonar data in response to activating the sonar-based movement sensing. The method may comprise detecting, by the mobile device, a breathing pattern in the sonar data. The method may comprise, in response to detecting the breathing pattern in the sonar data, collecting, by the mobile device, respiration data of a user.

Embodiments of such a method may include one or more of the following features: detecting the breathing pattern in the sonar data may comprise separately analyzing data for a plurality of distance ranges. Detecting the breathing pattern in the sonar data further may comprise detecting, within a distance range of the plurality of distance ranges, a frequency within a defined breathing frequency range. The sonar-based movement sensing may comprise outputting a plurality of ultrasonic chirps. Each ultrasonic chirp of the plurality of ultrasonic chirps may comprise multiple frequencies. Each ultrasonic chirp of the plurality of ultrasonic chirps may comprise multiple increases and multiple decreases in chirp magnitude. The method further may comprise outputting, by the mobile device, via a display of the mobile device, respiration data for the user based at least in part on the collected respiration data of the user. An average power of the mobile device used for sensing and collection of respiration data may be between 0.1 mW and 0.4 mW. Detecting the breathing pattern in the sonar data may comprise outputting a first plurality of sonar chirps at a first frequency of chirps. Collecting respiration data of the user may comprise outputting a second plurality of sonar chirps at a second frequency of chirps. The second frequency of chirps may be greater than the first frequency of chirps. Determining that the mobile device may be stationary may be based on acceleration data obtained from an accelerometer of the mobile device. The mobile device may be exclusively powered by battery. The mobile device may be selected from the group consisting of: a smartphone; a smartwatch; a gaming device; and a tablet computer.

In some embodiments, a mobile device is described. A mobile device may comprise a sound sensor. A mobile device may comprise a sound emitter. A mobile device may comprise a movement sensor. A mobile device may comprise a processing system, comprising one or more processors in communication with the sound sensor, the sound emitter, and the movement sensor. The processing system may be configured to determine that the mobile device may be stationary based on data from the movement sensor. The processing system may be configured to activate sonar-based movement sensing using the sound emitter in response to determining that the mobile device may be stationary. The processing system may be configured to receive sonar data from the sound sensor in response to activating the sonar-based movement sensing. The processing system may be configured to detect a breathing pattern in the sonar data. The processing system may be configured to create respiration data for a user in response to detecting the breathing pattern in the sonar data.

Embodiments of such a device may include one or more of the following features: the sound sensor may be a microphone used for voice-based communications, the sound emitter may be a speaker that outputs ultrasonic sound and may be used for voice-based communications, and the movement sensor may be an accelerometer. The mobile device further may comprise a cellular network interface. The mobile device may be a smartphone that communicates with a cellular network via the cellular network interface. The processing system being configured to detect the breathing pattern in the sonar data may comprise separately analyzing data for a plurality of distance ranges. The processing system being configured to detect the breathing pattern in the sonar data further may comprise the processing system being configured to detect, within a distance range of the plurality of distance ranges, a frequency within a defined permissible breathing range. The sound emitter may output a plurality of ultrasonic chirps. Each ultrasonic chirp of the plurality of ultrasonic chirps may include multiple frequencies. Each ultrasonic chirp of the plurality of ultrasonic chirps that may be output by the sound emitter may comprise multiple increases and multiple decreases in chirp magnitude. The device further may comprise an electronic display. The processing system may be further configured to output the respiration data for the user via the electronic display. An average power of the mobile device consumed to perform sensing and collection of respiration data may be between 0.1 mW and 0.4 mW.

In some embodiments, a sonar-based respiration monitoring system is described. The system may comprise a cloud-based server system. The system may comprise a sound sensor. The system may comprise a sound emitter. The system may comprise a movement sensor. The system may comprise a processing system. The sonar-based respiration monitoring system may be configured to determine that the movement sensor may be stationary based on data from the movement sensor. The system may be configured to activate sonar-based movement sensing using the sound emitter in response to determining that the movement sensor may be stationary. The system may be configured to receive sonar data from the sound sensor in response to activating the sonar-based movement sensing. The system may be configured to detect a breathing pattern in the sonar data. The system may be configured to create respiration data for a user in response to detecting the breathing pattern in the sonar data.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a situation during which opportunistic sonar-based health monitoring can be performed.

FIG. 2 illustrates a block diagram of a system that performs opportunistic sonar-based health monitoring.

FIG. 3A illustrates an embodiment of a sonar waveform that can be used for opportunistic sonar-based health monitoring.

FIG. 3B illustrates an embodiment of the frequency of a sonar waveform that can be used for opportunistic sonar-based health monitoring.

FIG. 4 illustrates an embodiment of a cascade model for power usage during opportunistic sonar-based health monitoring.

FIG. 5 illustrates an embodiment of a method for opportunistic sonar-based health monitoring.

### DETAILED DESCRIPTION

Passive sensing for vital sign monitoring, such as breathing rate or heartrate, can operate in the background after explicitly receiving the user's informed consent to perform passive monitoring. Once initially configured and consented to, passive monitoring may be significantly beneficial to a user—such monitoring may be performed without any specific request or input from the user, the user may be less likely to consciously or unconsciously take an action to affect the measurement, and the user may be more likely to have the user's health monitored in frequency of measurement or duration. Embodiments detailed herein can be used for passive, opportunistic health monitoring. These embodiments detect when an appropriate time for sonar-based measurement occurs prior to initiating sonar sensing, thus helping to reduce power consumption. Once initiated, low-power sonar sensing may be performed to see if a user's vital sign can be detected. If it can be detected, high-power sonar sensing is then performed to monitor the vital sign. This monitoring can continue for a time until, for example, the vital sign cannot be detected or the device is moved. Monitoring may then be performed again at some time in the future when the device is stationary and the vital sign can be detected.

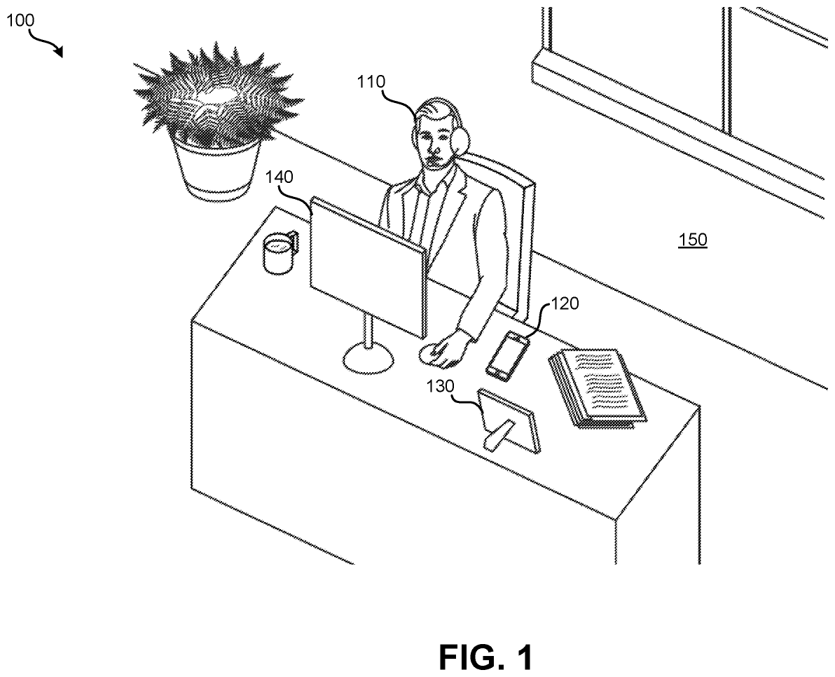

Further detail regarding these embodiments and other embodiments are provided in relation to the figures. FIG. 1 illustrates an environment 100 during which opportunistic sonar-based health monitoring can be performed.

In environment 100, user 110 is performing deskwork. The user has placed mobile device 120 on his desk. While sitting, user 110 may, at times, be effectively motionless except for one or more vital signs, such as breathing. Opportunistic sonar-based vital sign monitoring may be able to effectively isolate the vital sign of user 110 from reflections by static objects, such as picture frame 130, monitor 140, and wall 150. Further, such opportunistic sonar-based vital sign monitoring may only be performed when mobile device 120 is stationary and user 110 is sufficiently stationary. Therefore, opportunistic sonar-based vital sign monitoring may only be performed for short stints of time while the user is relatively motionless for some reason other than having his or her vital sign measured. For example, the user may be asleep or, as pictured, reviewing information on monitor 140. This form of vital sign monitoring is referred to as “opportunistic” because the user is not focused on having his or her vital sign being monitored—rather, the vital sign is monitored by mobile device 120 because the user and general environment happens to be in a state conducive to vital sign monitoring.

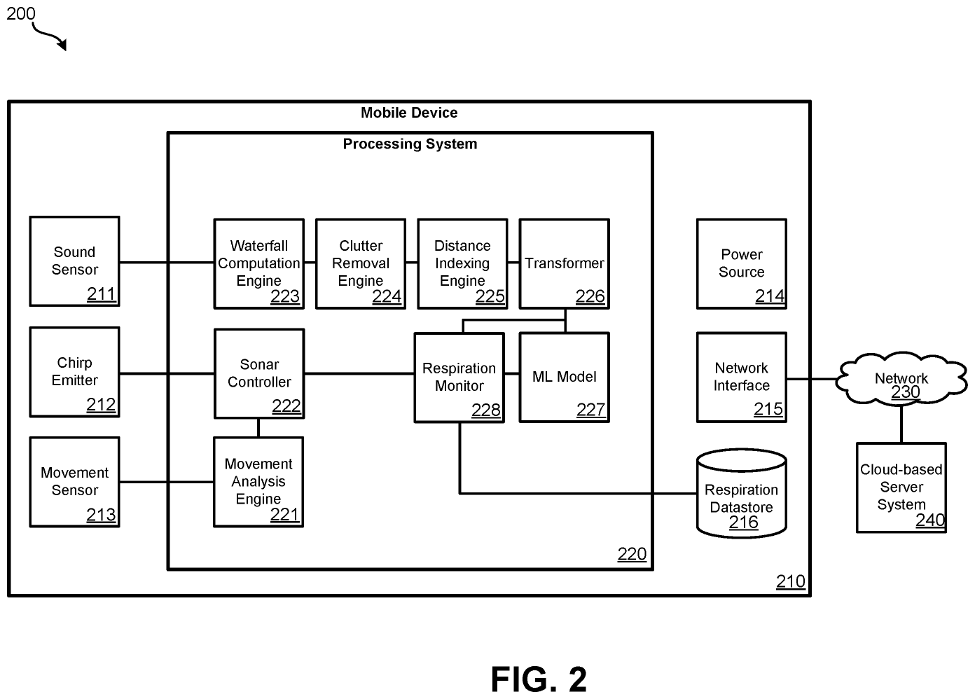

FIG. 2 illustrates a block diagram of an opportunistic sonar-based health monitoring system 200 (“system 200”). System 200 can be implemented as part of a mobile device, such as a smartphone, a cellular phone, a gaming device, a tablet computer, a smartwatch, laptop computer, earbuds, or some other form of computerized device. In some embodiments, system 200 can be implemented as part of a smart home device (that might not necessarily be mobile or may tend to be left in a particular location), such as a smart thermostat, smart home speaker, smart home hub, smart television, smart video camera, smart hazard detector (e.g., carbon monoxide and/or smoke), etc. “Smart” refers to a computerized device that is capable of communicating via a network.

Since the hardware (e.g., a microphone and speaker) required to perform opportunistic sonar-based health monitoring is present in most, if not all, smartphones, tablet computers, and gaming devices, specialized software or firmware may be distributed to a wide variety of computerized devices, such as with an operating system (OS) that operates on such computerized devices, or as an application (“app”) that can be downloaded by a user.

Mobile device 210 (or smart home device) can include: sound sensor 211; chirp emitter 212; movement sensor 213; power source 214; network interface 215; respiration datastore 216; and processing system 220. Additionally, mobile device 210 (or a smart home device) can include various other components, such as an electronic display (e.g., touchscreen), user input device (e.g., dial, buttons), one or more antennas, status lights, etc. For simplicity, such components are not illustrated on mobile device 210. For instance, the electronic display can be used to present respiration data to a user.

Sound sensor 211 may be one or more multipurpose microphones, which can be capable of receiving ultrasonic sound (i.e., sound above the auditory range of humans). In the case of some mobile devices, one or more such multipurpose microphones can also be used for capturing voice (e.g., for voice-based communications). In some embodiments, one or more dedicated sound sensors are present specifically for sonar-based use. Chirp emitter 212, which can be referred to as a sound emitter, may be a speaker that is capable of outputting ultrasonic sound. In some embodiments, multiple speakers may be used. The speaker may be a multipurpose speaker; the speaker may also be used for a speakerphone, outputting music, and/or as a phone speaker (e.g., for voice-based communications). In other embodiments, a dedicated chirp emitter 212 may be used that is exclusively used for sonar.

Movement sensor 213 can be one or more accelerometers that sense acceleration. Additionally or alternatively, other forms of movement sensors may be used, such as a gyroscopic sensor or, possibly, a global navigation satellite system (GNSS) sensor that can be used to determine if mobile device 210 is moving over time.

Power source 214 may be a battery or other portable power storage device, such as an ultra-capacitor. Therefore, for periods of time during use, a battery or some other portable power storage device may be used exclusively to power mobile device 210. For mobile devices and smart home devices that are not continuously connected to power, conservation of power may be especially important. Therefore, in such devices, continuous use of sonar may be avoided to decrease the amount of power consumed.

Network interface 215 may include one or more network interfaces that can communicate via wireless local area network protocols (e.g., WiFi) and/or mesh networking protocols (e.g., Thread, Zigbee). Both mobile devices and smart home devices may use such wireless networking protocols. Some smart home devices additionally or alternatively use a wired network connection (e.g., Ethernet). Network interface 215 may enable processing system 220 to communicate wirelessly or via a wired connection with network 230. Network 230 can include one or more public and/or private networks. A private network may be a home wireless local area network. A public network would typically be the Internet.

Network interface 215 may additionally or alternatively include a cellular network interface that allows for communication with a cellular network. A cellular network interface can permit communication with cellular networks that use one or more types of cellular radio access technologies (RATs), such as 4G LTE (Long Term Evolution), 5G NR (New Radio), or some other cellular network standard, such as 6G.

Network 230 may enable mobile device 210 to communicate with cloud-based server system 240. A user account may be maintained by cloud-based server system 240 that stores information obtained from mobile device 210. Additionally or alternatively, some of the functions detailed below as performed by processing system 220 can instead be performed in the cloud by cloud-based server system 240. Therefore, any or all functionality of components 221-228 may instead be performed by cloud-based server system 240.

Processing system 220 may include one or more special-purpose or general-purpose processors. Such special-purpose processors may include processors that are specifically designed to perform the functions detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random access memory (RAM), flash memory, or a solid state drive (SSD).

Processing system 220 performs various functions related to opportunistic sonar-based vital sign monitoring. These functions are represented as various components, which can be implemented using specialized software or specialized hardware. In other embodiments, the functionality of these components can be combined into fewer components or divided out into a greater number of components. In the illustrated embodiments, the components of processing system 220 include: movement analysis engine 221; sonar controller 222; waterfall computation engine 223; clutter removal engine 224; distance indexing engine 225; transformer 226; ML model 227; and respiration monitor 228. Further detail regarding each of these components is provided as follows.

Movement analysis engine 221 may receive movement data from movement sensor 213. If movement sensor 213 is an accelerometer, the data may be indicative of a magnitude and direction of acceleration. Movement analysis engine 221 may analyze received movement data to output an indication of whether movement sensor 213, and thus mobile device 210, are stationary or in motion. Mobile device 210 may be considered stationary when movement analysis engine 221 determines that a magnitude of acceleration is below a threshold magnitude, possibly for at least a threshold time period. Movement analysis engine 221 may output a state indication of whether mobile device 210 is determined to be moving or stationary. This state indication can be provided to or accessed by sonar controller 222.

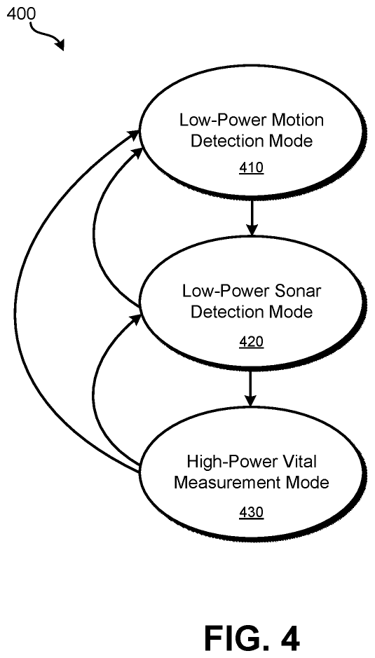

Sonar controller 222 controls whether chirp emitter 212 emits ultrasonic chirps and, possibly, at what rate. Sonar controller 222 may function in accordance with the state diagram of FIG. 4. Sonar controller 222 may receive an indication of whether mobile device 210 is likely moving from movement analysis engine 221. If in motion, sonar controller 222 may turn off sonar and chirp emitter 212 may not be caused to emit any ultrasonic chirps. When stationary, sonar controller 222 may determine a frequency at which chirp emitter 212 should emit ultrasonic chirps. The frequency may be based on whether or not a breathing signal has been successfully identified by respiration monitor 228: if no breathing signal is present, a lower rate frequency of chirps may be output than if a breathing signal is detected.

Other factors may also be factored in by sonar controller 222 in controlling whether and at what frequency ultrasonic chirps are output. The battery charge level of power source 214 may be a factor; if the charge level is below a threshold, opportunistic sonar-based health monitoring may be disabled. A user-editable setting may be a factor; a user may choose to temporarily or permanently disable opportunistic sonar-based health monitoring. Even if movement analysis engine 221 indicates mobile device 210 is stationary, if a breathing signal has not been detected within a certain amount of time, opportunistic sonar-based health monitoring may be disabled for some period of time. If a phone call, video playback, or audio playback is active, opportunistic sonar-based health monitoring may be disabled.

When activated by sonar controller 222, chirp emitter 212 may output ultrasonic chirps that are reflected off of one or more objects in an environment of mobile device 210, reflected, and received by sound sensor 211. Sonar controller 222 may control when an output of sound sensor 211 is monitored. For instance, when chirps are not being output and sound sensor 211 is not otherwise being used (e.g., as a microphone for a phone call), the output of sound sensor 211 may be ignored, such as for privacy and/or power usage reasons.

When sonar controller 222 is causing chirp emitter 212 to emit chirps, reflections of such chirps by objects in the ambient environment of mobile device 210 are received by sound sensor 211. The time delay between transmitted and received chirp pulses corresponds to the round-trip Time-of-Flight (ToF) between the sonar device and the object. The distance between the object, chirp emitter 212, and sound sensor 211 can be computed by Equation 1, where Vₛ is the speed of sound.

$$d = ToF * \frac{{V}_{s}}{2} \qquad Eq . 1$$

By determining d between the user and mobile device 210, the user's respiratory rate (or other vital sign) can be calculated by monitoring the change in distance (Δd) over time. To accomplish this calculation, a signal output by sound sensor 211 based on the received reflections of such chirps is converted from an analog signal to a digital signal and input to waterfall computation engine 223.

Waterfall computation engine 223 may serve to analyze the reflection of the emitted chirp, and compute a set of data for each chirp that is indicative of an amplitude of reflection received for various distances. Based on the speed of sound travelling through air and the amount of time that elapses between when a chirp is emitted and its reflection is received, the distance to the object that caused the reflection can be calculated. By emitting a series of chirps over time, waterfall computation engine 223 creates a multidimensional data stream that has a time dimension, a distance dimension, and an amplitude dimension.

More specifically, to create the waterfall signal, the transmitted chirp template can be cross-correlated with the received signal. Each peak in the correlation envelope corresponds to a ToF of a roundtrip path between chirp emitter 212, an environmental object (e. g., user's body, static object), and sound sensor 211. By using cross-correlation instead of frequency shifting, a matched filter is created, which can offer a higher SNR. Next, the envelope of the correlation signal may be computed with average pooling. The average pooling pools distances to various intervals. The range resolution can be defined by the bandwidth according to Equation 2:

$$Δ R = \frac{{V}_{s}}{2 Δ f} \qquad Eq . 2$$

Next, the correlation envelope of each chirp response is stacked to construct a waterfall data stream. Since each chirp's correlation contains the distance information of the environment objects, by stacking each chirp pulse's correlation envelope vertically, a two dimensional (plus amplitude) waterfall can be created. The waterfall's x-axis can correspond to time with the chirp rate instead of raw audio sampling rate, and the y-axis can correspond to distance. A magnitude value can also be present, for a total of three dimensions of data.

Clutter removal engine 224 analyzes the output waterfall data stream from waterfall computation engine 223. Clutter removal engine 224 serves to remove reflections from stationary objects. To remove reflections by stationary objects, clutter removal engine 224 may compare reflections of separate chirps over time. The static reflections can be removed by computing the difference between the reflection from the current chirp and the last chirp to create a delta waterfall. If an amplitude of reflection at a particular distance does not change, this is indicative that the reflection is off a stationary object and can be discarded. For example, if mobile device 210 is placed on a desk near a wall, reflections of chirps off the desk and wall are merely noise and are removed from the waterfall data prior to additional processing. A person exhibiting a vital sign, such as breathing or a heartbeat, will cause his or her body to move slightly in distance with each breath or heartbeat. This change in distance between mobile device 210 and the person's body is sufficient that portions of the person's body exhibiting the movement (e.g., chest) will not be removed as a stationary object by clutter removal engine 224. The filtered output of clutter removal engine 224 can then be passed to distance indexing engine 225.

Distance indexing engine 225 can serve to divide the filtered waterfall data into distance indexed waterfalls or distance indexed buckets. These distance indexed buckets may be created for various distance ranges, which may partially overlap or may not overlap. In some embodiments, hundreds of distance indexed waterfalls may be created. For example, each distance indexed waterfall may be for a distance between 1 and 2 cm, such as 1.4 cm. Therefore, the outputs of distance indexing engine 225 are multiple distance indexed waterfalls, with each of the waterfalls corresponding to a particular distance range.

Transformer 226 can perform a transform on each of the multiple distance indexed waterfalls. The transform performed by transformer 226 on each multiple distance indexed waterfall may be a continuous wavelet transform (CWT). In other embodiments, a short-time Fourier transform (STFT) may be performed or some other form of transform which calculates frequency from the amplitude, time, distance components of each of the multiple distance indexed waterfalls. Therefore, following transformer 226 performing the transform, for each of the multiple distance indexed waterfalls, a spectrum feature stream can be created that includes a frequency dimension, a time dimension, and an amplitude dimension. The spectrum feature stream for each of the multiple distance indexed waterfalls can then be analyzed by machine learning (ML) model 227.

CWT can be applied to the multiple distance indexed waterfalls to compute the signal's spectrogram. CWT is the convolution of the input signal with a set of functions generated by the mother wavelet at different scales. In some embodiments, the Morlet wavelet for the CWT spectrogram computation is used because it is closely related to human perception including both hearing and vision.

Comparing CWT and STFT, CWT can have certain advantages. Generally, CWT are more time-frequency neutral than STFT and, thus, are more suited to breathing, which is not perfectly periodic. More specifically, CWT can offer better time resolution than STFT. Since each wavelet transform reveals both the frequency information and the temporal information of the signal, while short-time FFT only reveals the frequency information of the short-time signal, CWT offers better time resolution than STFT. Further, flexibility in the frequency domain can be improved by using CWT. In CWT, the scales of the daughter wavelets control the spectrogram's frequencies and they can be self-defined. The CWT frequencies can be set to have a small resolution in the breathing frequency range and coarse resolution in other frequency ranges. In this way, the frequency details can be magnified in the breathing signals (or other vital signs) of interest and ignore frequencies in other signals (e.g., human motions). In some embodiments, a list of 60 CWT frequencies can be defined for a breathing rate implementation: the first 30 frequencies can be the breathing frequencies of interest, which start from 0.02 Hz (˜1 BPM) and linearly increase to 0.6 Hz (˜36 BPM) with a step of 0.02 Hz. The following 30 frequencies are the higher frequencies that correspond to motions, which start from 0.6 Hz and increase to 10 Hz, evenly spaced in log space.

ML model 227 can be a trained machine learning model that detects whether a user is motionless and breathing (or, some other vital sign which ML model 227 is trained to detect) in each spectrum feature stream. Therefore, the inputs to ML model 227, for each spectrum feature stream, are the same dimensions as the output of transformer 226: frequency, amplitude, and time. Each spectrum feature stream may be separately analyzed by ML model 227. The output of ML model 227 may be a score for each spectrum feature stream indicative of the probability that the spectrogram contains a valid respiratory signal. In some embodiments, the score for each stream ranges from 0 to 1. In other embodiments, the score ranges from −1 to 1, with 0 serving as a minimum threshold level for a valid respiratory signal.

ML model 227 may be a convolutional neural network (CNN) that has been trained using a set of training data mapped to ground truth data. The training data can consist of spectrum feature streams that have been truth-tagged with whether or not a human was actually present, motionless, and breathing (or whatever vital sign is being monitored) for creation of the training data. More specifically, the training data used to train ML model 227 can be samples of windowed sonar signals (e.g., 30 seconds in length) that have been manually truth-tagged as positive or negative for having a breathing signal present. The sonar signals used for training data can be recorded by smartphones in whole-night experiments and randomly extracted from multiple different users. Ground-truth data indicative of a subject's breathing rate can be gathered using a chest wrap that uses one or more accelerometers to measure the rise and fall of a subject's chest. The CNN can include two convolutional layers, each followed by a max-pooling layer, a flattened layer, and two fully connected layers. The last layer may use the sigmoid activation function, which outputs a probability between 0 and 1, which can be used as the confidence score.

A non-AI and non-ML implemented embodiment may alternatively be present. Instead, some other form of algorithm for checking whether vital sign frequencies is present in each spectrum feature stream of the multiple distance indexed waterfalls.

Depending on the embodiment, frequency-based filtering may be performed by ML model 227 itself or by another component. Frequencies above or below certain values can be excluded as potentially corresponding to a user's vital sign. For instance, the average breathing rate for a person is between 12 and 16 breaths per minute. Therefore, if the frequency detected is below a minimum (e.g., 6 BPM) or above a maximum (e.g., 25 BPM), the frequency data can be removed from the spectrum feature stream. In some embodiments, ML model 227 performs this function by virtue of having been trained using valid training data representative of humans' vital signs. Additionally or alternatively, this may be performed by transformer 226, as detailed in relation to the CWT.

Respiration monitor 228 can receive the scores from ML model 227 and determine which, if any, spectrum feature stream should be used for monitoring a vital sign, such as breathing, for a user. At most, a single spectrum feature stream may be selected. The spectrum feature stream selected may be the highest scored spectrum feature stream by ML Model 227. The score of the highest-scoring spectrum feature stream may be required to be above a minimum threshold. If it is not above the minimum threshold, a determination that a user's vital sign cannot be detected may be made. In some embodiments, the scores across multiple spectrum feature streams may influence whether a determination is made that a user's vital sign can accurately be detected. For instance, if many spectrum feature streams score above the minimum threshold, this may be an indication that significant noise and movement are present in the ambient environment that are unlikely to be just a user breathing. Again here, a determination that a user's vital sign cannot be detected may be made.

If respiration monitor 228 determines that the highest-scoring spectrum feature stream is above the minimum threshold and other spectrum feature streams have a significantly lower score, respiration monitor 228 may trigger sonar controller 222 to enter a high-power vital measurement mode, which involves a greater number of chirps being emitted and reflections analyzed. If respiration monitor 228 determines that the highest-scoring spectrum feature stream is not above the minimum threshold and/or other spectrum feature streams have a similar score, respiration monitor 228 may trigger sonar controller 222 to remain in a low-power vital measurement mode or may disable emission of chirps (e.g., for at least a defined period of time).

For a time window (such as 30 seconds), the respiratory rate may be computed by respiration monitor 228 by applying a fast Fourier transform (FFT) on the highest-scoring spectrum feature stream. The stream may be padded with some number of seconds (e.g., 45) of zeros on both sides, making it a total of, for example, 120 seconds. Each FFT bin can be defined by Equation 3:

$$\frac{Sampling Rate}{TotalSamples} \qquad Eq . 3$$

The frequency bin with the highest energy in the FFT results is taken as the user's respiratory rate. Only frequency ranges within a defined range, such as 8 BPM to 25 BPM, which covers the adult respiratory rate range, may be extracted and analyzed with other frequencies ignored. For adults, the normal respiratory rate is 12-20 BPM, and an adult with a respiratory rate of over 20 BPM is probably unwell.

When in the high-power vital measurement mode, based upon the frequency detected within the highest-scoring feature stream, respiration monitor 228 may store an indication of the user's measured vital sign, in this case, his or her respiration rate. This data may be stored to respiration datastore 216. Over time, this data may be collected and averaged (or otherwise combined) to determine the user's breathing rate, such as on a per day or per week basis. This respiration data can be output to the user or otherwise analyzed to identify potential health trends or problems. Additionally or alternatively, with the user's explicit permission, respiration data may be transmitted to cloud-based server system 240 for storage and/or analysis in association with a user account of the user.

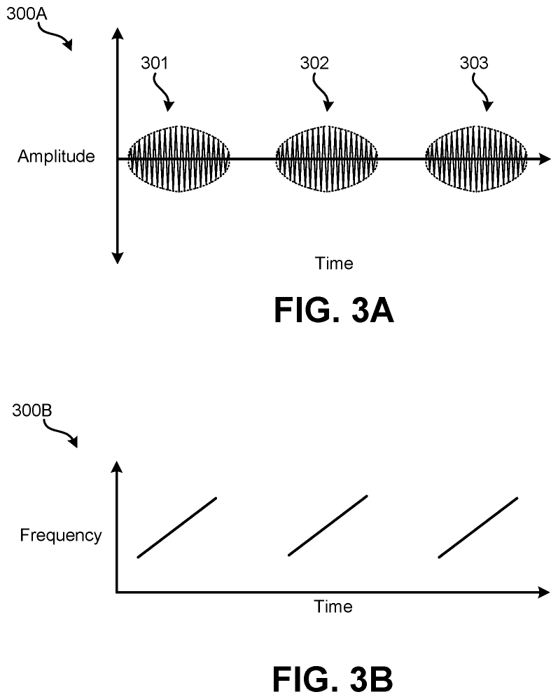

FIG. 3A illustrates an embodiment of a sonar waveform 300A that can be used for opportunistic sonar-based health monitoring. Sonar waveform 300A may be emitted by chirp emitter 212. As illustrated, three chirps are present: chirp 301, chirp 302, and chirp 303. A pure pulse signal may not be able to achieve the small range of resolution needed to detect a vital sign, such as breathing, while still maintaining an acceptable signal-to-noise (SNR) ratio.

Pulse compression can solve the range resolution and SNR trade-off using matched filtering. By implementing matched filtering, the original pulse can be compressed into a much narrower pulse in the correlation results. Thus, the compressed pulse achieves a smaller range resolution while maintaining the same SNR. The matched filtering can be implemented by modulating the transmitted pulse signal by chirp emitter 212 and correlating the known transmitted pulse with the received signal by sound sensor 211 to detect chirps. As shown in FIG. 3B in the frequency domain 300B, each chirp can be frequency modulated. In embodiments detailed herein, a frequency-modulated chirp pulse may be used, as detailed in Equation 2 and 3:

$$s ( t ) = {Ae}^{2 i π ( {f}_{0} + \frac{Δ f}{2 t} ) t} if - \frac{τ}{2} \leq t \leq \frac{τ}{2} \qquad Eq . 4$$

$$s ( t ) = 0 , otherwise \qquad Eq . 5$$

In Equations 4 and 5, f₀ is the lowest frequency, Δf is the frequency sweep range, and τ is the time length of the pulse. At the receiver side, the received signal is cross-correlated with the transmitted chirp pulse template by waterfall computation engine 223, and the cross-correlation of the transmitted signal s(t) and the received signal Ks(t), wherein K is the attenuation factor, can be written as below in Equations 6 and 7:

$$〈 s ( t ) , Ks ( t ) 〉 = {KA}^{2} τΛ ( \frac{t}{τ} ) sinc [ Δ ft Λ ( \frac{t}{τ} ) ] {e}^{2 i {π f}_{0} t} \qquad Eq . 6$$

$$wherein Λ ( \frac{t}{τ} ) = max ( 1 - \vert \frac{t}{τ} \vert ) , and sinc ( x ) = \frac{sin ( π x )}{π} \qquad Eq . 7$$

The correlation result shows that, after matched filtering, the resulting pulse resolution is the same as a pulse width length where

$${τ}^{′} = \frac{1}{Δ f} ,$$

which is usually much smaller than the transmitted pulse length τ. The ratio

$$\frac{τ}{{τ}^{′}} = τ * Δ f$$

is the pulse compression ratio. As an example, for a chirp pulse with a 20 ms pulse length and frequency range from 19 kHz to 21.5 kHz, the pulse compression ratio is 50. In some embodiments, the pulse compression ratio ranges from 20 to 100.

When emitting pulses or chirps or ultrasonic sound, discontinuities at the pulse edges cause spectral leakage and can make the transmitted ultrasound signal audible to humans at lower frequencies. A Hann windowing function can be applied to the pulsed chirp being output to filter out side lobes. As shown in FIG. 3A, the chirp pulse's waveform gradually decreases to zero at the start and end of each chirp, thus eliminating or at least significantly decreasing spectral leakage, especially into human's auditory range.

The frequency at which chirps are emitted may depend on the mode in which sonar controller 222 is set. In low-power sonar detection mode 420, which is used to determine if a breathing signal is present, chirps may be transmitted at a rate of 2 Hz or within the range of 1 Hz to 10 Hz. In high-power vital measurement mode, chirps may be transmitted at a rate of 20 Hz, or within the range of 10 Hz to 40 Hz. The higher the frequency, the greater the power consumption to emit the sound and process the reflections; therefore, keeping the frequency lower is beneficial for energy consumption reasons.

FIG. 3B illustrates an embodiment of the frequency of a sonar waveform that can be used for opportunistic sonar-based health monitoring. Over the course of each individual chirp, the frequency may be varied. For example, as illustrated in FIG. 3B, the frequency may be ramped up from the start to finish of each chirp. In some embodiments, the chirp pulses may range from 19 kHz to 21.5 kHz. In other embodiments, the chirp pulses may range from 26 kHz to 31 kHz. Stated more generally, the chirp pulses may range in frequency from 15 kHz (while people vary, 15 kHz is assumed to be the upper limit of an adult's auditory range) to 40 kHz. Since some people can hear sounds in the range of 15 kHz to 20 kHz, in some embodiments the minimum frequency emitted may be 18 kHz, 19 kHz, 20 kHz or higher. In other embodiments, the frequency may be decreased in a ramp function over the course of each chirp or varied in some other pattern.

FIG. 4 illustrates an embodiment of cascade model 400 for power usage during opportunistic sonar-based health monitoring. Cascade model 400 may represent the three power modes in which opportunistic sonar-based health monitoring can operate. If movement is occurring or has recently occurred (e.g., within a threshold amount of time), sonar-based health monitoring may operate in mode 410. In mode 410, no sonar sensing may be performed for sonar-based health monitoring. Rather, the accelerometer or other form of movement sensor may be monitored periodically, such as at a rate of 10 Hz or between 1 Hz and 30 Hz. Mode 410 may be understood as using no additional power (0 mW) because the mobile device can be expected to already be performing such movement sensing for other purposes, such as activating a display.

Mode 410 may transition to mode 420 when the movement sensor indicates that the mobile device is static, possibly for at least the threshold period of time. In mode 420, low-power sonar detection is performed. In mode 420, sonar-sensing is performed at a low frequency, such as emission of chirps and evaluation of reflected chirps at 2 Hz, or between 1 Hz and 10 Hz. Mode 410 may consume 3 mW, or in other embodiments, between 2 mW and 6 mW. This low frequency is sufficient to detect whether breathing (or perhaps some other vital sign) is present or not, but may not be sufficient to accurately measure the vital sign. If movement is sensed by the movement sensor while in mode 420, mode 420 may transition back to mode 410. If breathing is detected, mode 420 may transition to mode 430. If no breathing is detected, the sonar-based health monitoring system may remain in mode 420 for at least a period of time to continue attempting to detect breathing. If not breathing is detected for an extended time period in mode 420, low-power sonar sensing may be suspended to preserve power.

In mode 430, high-power vital measurements may be performed. Mode 430 can involve emission of chirps and evaluation of reflected chirps at a frequency of 20 Hz or between 10 Hz and 30 Hz (a higher frequency may be used for other vital signs, such as heartrate). Mode 430 can involve emitting sonar chirps at the highest frequency, and, thus the greatest amount of power is consumed. Power consumption in mode 430 can be 30 mW or, in other embodiments, between 10 mW and 50 mW. Mode 430 may remain active while a vital measurement is successfully being made. If breathing (or some other vital sign) can no longer be detected, mode 430 may transition back to mode 420. If the mobile device begins moving, mode 430 may transition back to mode 410.

The average power consumption of opportunistic sonar-based health monitoring can be based on the average amount of time that the device spends in each mode. Therefore, assuming power usage of 0 mW in mode 410, 3 mW in mode 420, and 30 mW in mode 430, the average power consumption may be 0.3 mW, or between 0.1 and 0.5 mW. The amount of power used will vary based on the amount of time that the mobile device spends in each mode; the previous example of 0.3 mW assumes that mode 420 is entered from mode 410 20% of the time, and mode 430 is entered from mode 420 only 10% of the time. These numbers have been estimated as average based on empirical studies.

In other embodiments, it may be possible to perform sonar-based health monitoring using more than three modes. Alternatively, in some embodiments, only two sonar-based health monitoring modes may be used: a low-power motion detection mode and a vital measurement mode.

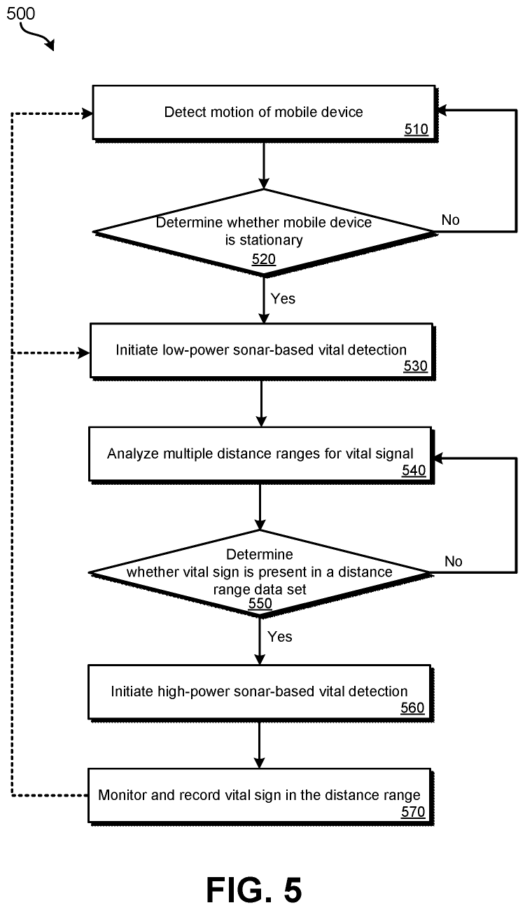

Various methods may be performed using the systems, arrangements, chirp structures, and modes detailed in relation to FIGS. 1-4. FIG. 5 illustrates an embodiment of a method 500 for opportunistic sonar-based health monitoring. Method 500 may be performed using system 200 of FIG. 2. Method 500 may also involve use of chirps as detailed in relation to FIGS. 3A and 3B. Method 500 may involve the use of modes as detailed in relation to FIG. 4. Prior to block 510, permission may be obtained from a user to perform monitoring of one or more vital signs, such as breathing rate or heart rate. Without permission being granted, method 500 may not be performed. For the remainder of method 500, the vital sign monitored is assumed to be breathing rate.

At block 510, motion of a mobile device may be detected. Such motion detection may be performed using one or more accelerometers or some other form of motion sensor that may be incorporated as part of the mobile device. If a smart home device is used to perform method 500, blocks 510 and 520 may not be performed because the smart home device is expected to be stationary, such as a smart home speaker. Block 510 can involve acceleration or movement measurements or data being transmitted to a processing system for evaluation.

At block 520, a determination may be made, based on the movement measurements or data, as to whether the mobile device is sufficiently stationary. This determination can involve movement measurements being indicative of zero movement or below a threshold of movement, possibly for at least a duration of time. For example, the mobile device may be required to be motionless for twenty seconds to be indicative of the mobile device being placed on a desk, such as illustrated in FIG. 1. If the mobile device is not stationary, motion of the mobile may be continued to be monitored and evaluated at blocks 510 and 520.

If the mobile device is determined to be (sufficiently) stationary, method 500 may proceed to block 530. At block 530, sonar-based vital detection may be initiated. In some embodiments, this can involve sonar-vital detection occurring in a low power mode, such as detailed in relation to mode 420. Low-power sonar-based detection may be performed at a sufficient frequency to determine whether the user is present and breathing, but not at a high enough frequency to accurately measure the breathing rate. In other embodiments, a single sonar-based mode may be present that is performed at a sufficient frequency to detect whether breathing is present and the breathing rate.

At block 540, analysis of reflections of chirps may be performed as detailed in relation to components 223, 224, 225, 226, 227, and 228 of FIG. 2. The analysis of block 540 can involve analyzing reflections for various distance ranges or bins and determining the distance range or bin at which breathing is most-likely present at block 550. If breathing is not detected with a sufficient confidence, method 500 may return to block 540 and continue monitoring for breathing, possibly in the low-power sonar-based mode.

If breathing is detected at block 550 with a sufficient confidence, method 500 may proceed to block 560. At block 560, if multiple sonar-based detection modes are used, the device may transition to a high-power sonar-based vital detection mode. This mode can involve greater power usage and emission of chirps at a higher frequency that is sufficient to accurately measure the vital sign, such as breathing rate.

At block 570, the vital sign can be monitored within the distance range or bin identified at block 550. Measurements of the vital sign may be stored and averaged together, such as to determine an average breathing rate over time (e.g., average breathing rate for a day). If the device is moved, method 500 can return to block 510 and high-power sonar-based vital detection may be stopped. If breathing can no longer be sensed with a high enough confidence in the distance range or bit, method 500 can return to block 530 to monitor for breathing in the low-power sonar-based vital detection mode.

It should be noted that the methods, systems, and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known, processes, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, it is noted that the embodiments may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

What is claimed is:

1. A method for sonar-based respiration monitoring, the method comprising:
determining, by a mobile device, that the mobile device is stationary;
in response to determining that the mobile device is stationary, activating, by the mobile device, sonar-based movement sensing;
capturing, using a sonar sensor of the mobile device, sonar data in response to activating the sonar-based movement sensing;
detecting, by the mobile device, a breathing pattern in the sonar data; and
in response to detecting the breathing pattern in the sonar data, collecting, by the mobile device, respiration data of a user.

2. The method for sonar-based respiration monitoring of claim 1, wherein detecting the breathing pattern in the sonar data comprises separately analyzing data for a plurality of distance ranges.

3. The method for sonar-based respiration monitoring of claim 2, wherein detecting the breathing pattern in the sonar data further comprises detecting, within a distance range of the plurality of distance ranges, a frequency within a defined breathing frequency range.

4. The method for sonar-based respiration monitoring of claim 1, wherein the sonar-based movement sensing comprises outputting a plurality of ultrasonic chirps, wherein each ultrasonic chirp of the plurality of ultrasonic chirps comprises multiple frequencies.

5. The method for sonar-based respiration monitoring of claim 4, wherein each ultrasonic chirp of the plurality of ultrasonic chirps comprises multiple increases and multiple decreases in chirp magnitude.

6. The method for sonar-based respiration monitoring of claim 1, further comprising:
outputting, by the mobile device, via a display of the mobile device, respiration data for the user based at least in part on the collected respiration data of the user.

7. The method for sonar-based respiration monitoring of claim 1, wherein an average power of the mobile device used for sensing and collection of respiration data is between 0.1 mW and 0.4 mW.

8. The method for sonar-based respiration monitoring of claim 1, wherein:
detecting the breathing pattern in the sonar data comprises outputting a first plurality of sonar chirps at a first frequency of chirps; and
collecting respiration data of the user comprises outputting a second plurality of sonar chirps at a second frequency of chirps, wherein
the second frequency of chirps is greater than the first frequency of chirps.

9. The method for sonar-based respiration monitoring of claim 8, wherein determining that the mobile device is stationary is based on acceleration data obtained from an accelerometer of the mobile device.

10. The method for sonar-based respiration monitoring of claim 1, wherein:
the mobile device is exclusively powered by battery; and
the mobile device is selected from the group consisting of: a smartphone; a smartwatch; a gaming device; and a tablet computer.

11. A mobile device, comprising:
a sound sensor;
a sound emitter;
a movement sensor; and
a processing system, comprising one or more processors in communication with the sound sensor, the sound emitter, and the movement sensor, the processing system is configured to:
determine that the mobile device is stationary based on data from the movement sensor;
activate sonar-based movement sensing using the sound emitter in response to determining that the mobile device is stationary;
receive sonar data from the sound sensor in response to activating the sonar-based movement sensing;
detect a breathing pattern in the sonar data; and
create respiration data for a user in response to detecting the breathing pattern in the sonar data.

12. The mobile device of claim 11, wherein the sound sensor is a microphone used for voice-based communications, the sound emitter is a speaker that outputs ultrasonic sound and is used for voice-based communications, and the movement sensor is an accelerometer.

13. The mobile device of claim 12, the mobile device further comprising a cellular network interface, wherein the mobile device is a smartphone that communicates with a cellular network via the cellular network interface.

14. The mobile device of claim 11, wherein the processing system being configured to detect the breathing pattern in the sonar data comprises separately analyzing data for a plurality of distance ranges.

15. The mobile device of claim 14, wherein the processing system being configured to detect the breathing pattern in the sonar data further comprises the processing system being configured to detect, within a distance range of the plurality of distance ranges, a frequency within a defined permissible breathing range.

16. The mobile device of claim 11, wherein the sound emitter outputs a plurality of ultrasonic chirps, wherein each ultrasonic chirp of the plurality of ultrasonic chirps includes multiple frequencies.

17. The mobile device of claim 16, wherein each ultrasonic chirp of the plurality of ultrasonic chirps that is output by the sound emitter comprises multiple increases and multiple decreases in chirp magnitude.

18. The mobile device of claim 11, further comprising an electronic display, wherein the processing system is further configured to output the respiration data for the user via the electronic display.

19. The mobile device of claim 11, wherein an average power of the mobile device consumed to perform sensing and collection of respiration data is between 0.1 mW and 0.4 mW.

20. A sonar-based respiration monitoring system, comprising:
a cloud-based server system;
a sound sensor;
a sound emitter;
a movement sensor; and
a processing system, wherein the sonar-based respiration monitoring system is configured to:
determine that the movement sensor is stationary based on data from the movement sensor;
activate sonar-based movement sensing using the sound emitter in response to determining that the movement sensor is stationary;
receive sonar data from the sound sensor in response to activating the sonar-based movement sensing;
detect a breathing pattern in the sonar data; and
create respiration data for a user in response to detecting the breathing pattern in the sonar data.
